(19) United States
(12) Patent Application Publication
(10) Pub. No.: US 2009/0092570 A1
(43) Pub. Date: Apr. 9, 2009

(54) ODOR-CONTROLLING COMPOSITION

(76) Inventors: Chrystine Richardson, (US); Lee Hanson, (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/247,988

(22) Filed: Oct. 8, 2008

Related U.S. Application Data
(60) Provisional application No. 60/978,332, filed on Oct. 8, 2007.

(51) Int. Cl. A61L 9/01

(52) U.S. Cl. 424/76.1

(57) ABSTRACT

A composition for odor reduction and odor elimination and methods for reducing and eliminating odors and preparing the composition.

### CROSS-REFERENCE TO RELATED APPLICATIONS

This application is a continuation of U.S. Patent Application No. 60/978,332, filed on Oct. 8, 2007, which is incorporated herein by reference.

### BACKGROUND OF THE INVENTION

1. Technical Field

This invention relates to compositions for reducing and eliminating odors and to methods for reducing and eliminating odors.

2. Prior Art

Most air fresheners and odor eliminators rely on fragrances to counteract the foul-smelling rancidity associated with biological and organic decomposition. However, fragrances in air fresheners only provide limited odor control, as they offer only short-term masking of the offensive odors. More specifically, fragrances have little or no long-lasting effect on odors attributable to the decay of organic matter, which includes low-molecular weight fatty acids, mercaptans, amines, indols, ammonia, and hydrogen sulfide.

Accordingly, there is always a need for improved methods and compositions for reducing or eliminating odors. It is to this need, among others, that this invention is directed.

### SUMMARY

This invention relates to compositions and methods for reducing or eliminating odors emitted by an odorous substrate. In one specific embodiment, the odor-controlling composition includes spore forming bacteria including thermophilic Bacillus spores which can germinate and produce effective amounts of enzymes, an effective amount of an odor-trapping material, an effective amount of a surfactant (for product stability), and effective amounts of a buffer, fragrance, and/or preservative. It is understood that various impurities can be present in the odor-controlling composition. Another specific embodiment include a method of preparing or producing the odor reducing composition.

Another specific embodiment includes a method of reducing or eliminating odors of an odorous substrate by applying to the odorous substrate an odor-controlling composition comprising spore forming bacteria.

These features and other features will become more apparent to those of ordinary skill in the relevant art when read with the following detailed description.

### DETAILED DESCRIPTION

### Definitions

In this specification, various terms are defined as follows:

The term “effective amount” means a level of an ingredient sufficient to effect the purpose of the ingredient. For illustration, an “effective amount” of an odor-controlling composition would mean an amount sufficient to absorb odor to the point that it is less noticeable. For further illustration, an “effective amount” of a preservative would be an amount of the preservative sufficient to prevent spoilage, or prevent microorganism growth in the composition.

The term “environmental malodors” includes any odor that is not the result of human or mammalian body odor and/or body fluids. For example, such odors include odors from foods, such as garlic, onions, and spices; cooking; smoke; tobacco; and gasoline.

The term “odor eliminating” means the removal of odors. The term “eliminating”, in the context of odors, is defined as lessening the intensity, duration, and offensiveness of an odor or smell such that the odor or smell is at least marginally eliminated.

The term “odor reduction” means the reduction of odors.

The term “controlling odor” means reducing and/or eliminating odor.

The term “removing”, in the context of odors, is herein defined as eliminating or ending the intensity, duration, and offensiveness of an odor or smell.

Unless otherwise defined, all technical and scientific terms used have the same meaning as commonly understood by one of ordinary skill in the art to which this invention pertains. Although methods and materials similar or equivalent to those described herein can be used in the practice and testing of this invention, suitable methods and materials are described below. In addition, the materials, methods, and examples are illustrative only and not intended to be limiting.

### SPECIFIC EMBODIMENTS

Embodiments of this invention include compositions and methods for reducing or eliminating odors emitted by substrate. A substrate that can be treated by embodiments of this invention include fecal matter, vomit, urine, or sewage sludge, which can create environmental malodors. Further, the odors emitted can be from bacterial digestions of fats, oils, greases, starches, proteins, e.g., in soil, compost, animal waste, diapers, garbage, food waste, and septic tanks. It is understood that this embodiment can be applied to a substrate having hydrocarbons, nitrogens, phosphates and/or micronutrients.

One specific embodiment of this invention relates to an odor-controlling composition comprising a spore forming thermophilic Bacillus. The odor-controlling composition comprising:

a) an effective amount to a spore forming thermophilic Bacillus, which can range from about 0.001% to about 2.0% by weight of the composition;

b) an effective amount of an odor-trapping material (e.g. zinc recinoleate), which can range from about 0.02% to about 3.0% by weight of the composition;

c) an effective amount of a surfactant (e.g. Calsoft L-40, tomadol 91-6, Macam MEJ, etc.), which can range from about 0.1% to about 5.0% by weight of the composition;

d) an effective amount of metallic salt (e.g. zinc, magnesium, calcium, etc.) from about 0.03 ppm to about 16.0 ppm by weight of the composition; and

e) effective amounts of a buffer, fragrance, and/or preservative, which can range from about 0.03% to about 3.0% by weight of the composition. All percentages are approximate, and are percent by weight. Other inert substances may be included in the composition, provided the chemistry or the biology of the product remains intact.

In one specific embodiment, the suitable microorganisms are strains of spore-forming Bacillus or other spore-forming microorganisms having the ability to reproduce at higher temperatures, e.g., at 55 degrees Celsius or above or e.g., 45 degrees Celsius or above. Such strains include, but are not limited to, Bacillus amyloliquefaciens, Bacillus stearothermophilus Bacillus licheniformis or Bacillus caldolyticus. In one example, Bacillus amyloliquefaciens was used to prepare an effective composition. The amount of microorganisms added to the composition can be mostly dependent on cost and solubility of the bacteria.

Further, the odor-controlling composition also can include enzymes, which are designed to digest proteins and other biomaterial. While bacteria can synthesize numerous different enzymes (which can be unique to the food source encountered), the enzyme for use with this embodiment can be selected from the protease group comprising amylase, protease, cellulase, lipase, xylanase, and genetic variations thereof.

An odor-trapping material, such as zinc recinoleate or a divalent ion recinoleate, also can be included in the odor composition. In one example, this molecule can be derived from castor oil and can act to counter environmental malodors. It is thought that zinc recinoleate can bind malodors covalently, which can cause aggregation of substrates. The percentage or amount of this material in the composition can be limited by its solubility within the composition. In one example, the concentration was about 10% of an active solution. It is understood that a higher amount of zinc recinoleate or divalent recinoleate in the composition can improve the deodorizing capability of the composition.

A fragrance also can be added to the odor-controlling composition. For example, it has been found that at least two fragrances, namely, herbal and fresh, were suitable with the invention. While both essence oil (a non-synthetic fragrance) and non-essence oil fragrances can be included in the composition, essence oil (e.g. orange oil or lemon oil) can be desired when the composition is marketed as a more natural product.

To add stability, a surfactant can be added to the composition. Surfactants are known in the art of forming oil-in-water emulsions. More suitable surfactants do not have a high level of interaction with the spore component of the composition. The surfactants can help optimize both the odor-absorbing characteristic of composition and the blending ability of composition. Surfactants suitable with this embodiment include surfactants able to stabilize the formula (hydrotrope of sorts). In many cases, the surfactant system may not directly participate in the odor control but can provide stability to the formula.

The composition also can include a buffer system in the composition to maintain the pH at more desired levels (e.g. at a pH from about 4.5 to about 10) for the bacteria. In one embodiment, the composition was buffered at a pH of about 7.

In one specific example, conventional buffering agents in the prior art could be used to adjust the pH to the desired level. For example, combinations of salts and acids, such as the following examples: potassium phosphate, lactic acid, citric acid, phosphoric acid, hydrochloric acid and sodium hydroxide, could be used. The amount can be very small and can be an amount to obtain the buffering effect. One of ordinary skill in the art can select an appropriate buffering system or combinations of agents without undue experimentation.

A preservative also can be added to the odor-controlling composition. A suitable preservative system can be used to control the percentages of extraneous microorganisms. It also can control the spores in the product so that they do not undergo early germination prior to application. In one example, the preservative becomes inert, once the composition is applied, so that the bacteria can degrade the substrate. It is possible to use more than one preservative to maintain lower levels of extraneous microorganisms.

The compositions also can contain adjunct odor-controlling materials, such as divalent salts, water-soluble cationic polymers, water-soluble anionic polymers, water-soluble carbonate salts, water-soluble bicarbonate salts, zeolites, and activated carbon; chelating agents; and/or colorants.

For example, zinc is a divalent cation, meaning that it is an ion that can bind to two different molecules, or one molecule. When a culture is fortified with zinc, the zinc can bind to the carboxyl groups, amino groups and dipicolinc acid contained by the peptidoglycan cell wall of the spore coat. Zinc ricinoleate can be added and can bind to this divalent metal ion. Other types of zinc molecules (e.g. zinc sulfate heptahydrate) can be used with embodiments of this invention.

Further, other divalent cations also may be used and included in the composition. For example, magnesium, manganese, iron, or even calcium can be included in the composition. One of ordinary skill in the art may use other cations with this invention without undue experimentation.

To improve the dispensing of the composition, the various ingredients can be combined with volatile and non-volatile volatile solvents, solubilizing agents, chemical carriers, chelators, and colorants to form a liquid that can be atomized into a fine mist spray or that can be wiped on a surface. In many cases, the bacteria should be mixed sufficiently to aid in dispersal so to help break up the clumps of spores.

In application and use, the odor-controlling composition can be applied to substrate emitting the odor, after which the spore can germinate to shed the spore coat and become vegetative (e.g. in about 2 to 8 hours), which can vary depending on species, temperature, moisture, pH and nutrients available (e.g. a carbon source can trigger growth). In one example, it was found that the spore forming bacteria activated at the lower pH environment of the substrate environment. The vegetative bacteria then can degrade the substrate and the other components and can also act on the substrate to achieve even further odor control. It may be possible to achieve improved results by maintaining a moist environment.

After composition has accomplished its purpose, it may not be necessary to clean up the composition. In one case, the spores in the composition can sporulate (and remain in area) and later re-germinate if the organic materials reappear. Alternatively, the vegetative cells that do not sporulate may die off or lyse. In these cases, the microbe can then become virtually undetectable (even microscopically).

Another embodiment includes a method for eliminating or neutralizing odor, comprising applying a thermophillic bacillus spore to an odiferous substrate.

In some studies, it was found that that there was little or no toxicity and side effect associated with specific embodiments of this invention. In most cases, the odor eliminating composition could be used as a reliable countermeasure for treatment of offensive odor in environments such as bathrooms and gyms. If the microorganisms in composition are not pathogenic, it is possible to us high levels or larger amounts of the composition without toxicity. Further, the microorganism content can be controlled by the removal or degradation of the waste (nutrition) or by applying bleaching to the composition. The limiting factor for their growth can be the complete digestion of the waste.

It is understood that the continuous spray or administration of the composition can be to suppress the generation of offensive odor.

### EXAMPLE 1

In one example, the composition was prepared in a batch kettle with a charge fermentor with prepared medium water by adding and mixing the following dry components.

| COMPONENT | g/L |
|---|---|
| Nitrogen | 10.0 |
| source | |
| Carbohydrates | 33.3 |
| Protein | 10.0 |
| Source | |
| Trace | 11.0 |
| minerals | |
| ZnSO4—7H2O | 0.014 |
| Antifoam | ~0.5 |
| Deionized | 935 mL |
| water | |

Once the fermentor was sterile and was stabilized at the optimal fermentation temperature, the air, the pH, the agitation and the backpressure were adjusted to a desired levels. The microorganisms (Bacillus amyloliquefaciens, Bacillus stearothermophilus Bacillus licheniformis or Bacillus caldolyticus) then were added to the culture. After microorganisms grew to a stage of 95% endospores, 0.014 g/L of ZnSO₄ was added to the mixture. The culture was processed further, e.g. according Example 2 (below), into an odor-controlling composition.

### EXAMPLE 2

The culture was fortified during the endospore phase with the amount of divalent cations used in the media formula for the initial batch. Adding the cations at this point in the process was found to help prevent overloading the culture with material, and it also was found to save the cations for use in its self-preservation state rather than its highly metabolic state. When the cations were used at this endospore phase, the spores take up the cations and use them to make a “cement” like spore coat. This phenomenon was found to work with thermophilic types of bacteria. The cations were injected when the fermentation broth revealed approximately 95% endospores.

The bacteria were grown by a method of batch fermentation. That way all of the culture was at roughly the same age and maturation level throughout the duration of the fermentation. The growth curve for this particular bacterial culture is sigmoidal. The culture is fortified with the cations close to the point of inflection in the curve.

The foregoing written description relates to various embodiments of the present invention. Numerous changes and modifications may be made therein without departing from the spirit and scope of the invention as defined in the following claims.

What is claimed is:

1. The odor-controlling composition comprising:
a) an effective amount to a spore-forming bacteria;
b) an effective amount of an odor-trapping material;
c) an effective amount of a surfactant; and
d) an effective amount of metallic salt.

2. The composition as claimed in claim 1, wherein the effective amount of the spore-forming bacteria is between about 0.001% to about 2.0% by weight of the composition.

3. The composition as claimed in claim, wherein the spore-forming bacteria is thermophillic bacteria.

4. The composition as claimed in claim 1, wherein the spore-forming bacteria is selected from the group consisting bacillus.

5. The composition as claimed in claim 1, wherein the spore-forming bacteria is Bacillus amyloliquefaciens.

6. The composition as claimed in claim 1, wherein the effective amount of an odor-trapping material is between about 0.02% to about 3.0% by weight of the composition.

7. The composition as claimed in claim 1, wherein the effective amount of the surfactant is between about from about 0.1% to about 5.0% by weight of the composition.

8. The composition as claimed in claim 1, wherein the effective amount of the metallic salt is from about 0.03 ppm to about 16.0 ppm by weight of the composition.

9. The composition as claimed in claim 1, wherein the metallic salt.

10. The composition as claimed in claim 1, wherein the metallic salt is composed of a divalent metal cation.

11. The composition as claimed in claim 1, wherein the metallic salt is selected from group consisting of zinc, magnesium, calcium, copper and combination thereof.

12. The method as claimed in claim 1, wherein the effective amount of an odor-trapping material is a recinoleate.

13. A method for reducing odors in an environment, comprising:
a) obtaining a composition comprising an effective amount to a spore-forming bacteria, an effective amount of an odor-trapping material, an effective amount of a surfactant, an effective amount of metallic salt; and
b) applying the composition to a substrate emitting the odor for a period of time;
wherein the application of the composition activates the bacteria, which then degrades a part of the substrate.

14. The method as claimed in claim 13, further comprising:
c) removing the composition after the period of time.

15. The method as claimed in claim 13, wherein the metallic salt is a divalent salt.

16. The method as claimed in claim 13, wherein the spore-forming bacteria is thermophillic bacteria.

17. The method as claimed in claim 13, wherein the spore-forming bacteria is selected from the group consisting bacillus.

18. The method as claimed in claim 13, wherein spore-forming bacteria is Bacillus amyloliquefaciens.

19. A method for preparing an odor reducing composition, comprising:
a) selecting a spore-forming bacteria and an odor-trapping material;
b) combining the bacteria and the odor-trapping material;
c) adding a surfactant;
d) adding metallic salt; and
e) adjusting the ph to about 7.

20. The method as claimed in claim 19, wherein spore-forming bacteria is Bacillus amyloliquefaciens.
